(19) United States
(12) Patent Application Publication
(10) Pub. No.: US 2024/0426017 A1
(43) Pub. Date: Dec. 26, 2024

(54) MICRONEEDLES AND METHOD FOR THEIR MANUFACTURE

(71) Applicant: IQS GROUP s.r.o., Husinec-Rez (CZ)

(72) Inventors: Tomas Tethal, Husinec-Rez (CZ); Stepan Cerny, Husinec-Rez (CZ)

(73) Assignee: IQS GROUP s.r.o., Husinec-Rez (CZ)

(21) Appl. No.: 18/684,724

(22) PCT Filed: Aug. 8, 2022
(86) PCT No.: PCT/EP2022/072236

§ 371 Date: Feb. 19, 2024

(30) Foreign Application Priority Data
Aug. 20, 2021 (GB) 2111971.4

(51) Int. Cl. C25D 7/00; C25D 5/54

(52) U.S. Cl. CPC C25D 7/00; C25D 5/54

(57) ABSTRACT

A method for the manufacture of one or more (especially hollow) microneedles (16), e.g. in the form of an array, patch or chip of side-by-side arranged microneedles, e.g. for delivering a pharmaceutical composition to the skin of a subject over an area thereof, the method comprising: (i) providing a substrate structure (14) of electrically insulating material including one or more concavities (12) extending into the body of the substrate structure (14) from a face thereof, the one or more concavities (12) being internally shaped and configured so as to correspond to the external shape and configuration of the respective one or more microneedles (16) to be formed; (ii) applying selectively to at least one first surface or surface portion of each of the said one or more concavities (12), and especially not in lower open-tip-forming regions 18 thereof, a layer (8) of an electrically conductive material; and (iii) using a galvanoplastic technique, depositing on each layer (8) of electrically conductive material in the said one or more concavities (12) at least one layer or body of microneedle wall-forming material (16) so as build up a wall (16) of a respective microneedle in each respective said concavity (12).

### TECHNICAL FIELD

This invention relates to a method for the manufacture of microneedles, and to microneedles manufactured or formed thereby. More particularly, though not exclusively, the invention relates to a method for the manufacture or formation of microneedles, especially hollow microneedles, more especially one or more (or, in some forms, a plurality of) hollow microneedles formed as or presented in the form of an array or chip or patch thereof, which in some forms may possibly contain up to a large number of such microneedles.

### BACKGROUND OF THE INVENTION AND PRIOR ART

Hollow needles of various physical types and sizes are commonly used to deliver a wide range of active substances to another, often solid, environment. A typical example is the delivery of a liquid pharmaceutical (or other active substance-containing) composition to the human or animal body, such as the injecting of a vaccine to a human subject during a vaccination procedure. For some applications, it may be necessary or more efficient to apply the active substance only just below the surface of the skin of the recipient, and for such applications it is common nowadays to employ microneedles for this purpose. Due to their small individual sizes (e.g. typically of the order of around ˜150-1500 μm long, ˜50-250 μm wide and with ˜1-25 μm sharp tip widths), microneedles are typically manufactured and provided for use in the form of an array or matrix, e.g. in the form of a patch or “chip”, containing a large number of adjacent microneedles formed in situ on a suitable substrate or carrier, which microneedle array is thus able to deliver a required overall dose of an active composition by collective delivery of individual amounts thereof from the individual microneedles of the array substantially simultaneously.

A wide range of different microneedle types are known in the art, and may be definable for example in terms of the physical nature or structure of their substrate/carrier (e.g. solid, swellable, etc), or in terms of the material of which the microneedles are composed (e.g. metal, ceramic, glass, polymer, etc), or in terms of the physical nature or structure of the microneedles themselves and the manner in which the active composition is transported and delivered to the desired target site or location in the recipient (e.g. by virtue of the microneedles being hollow (but solid-walled), solid (but coated), dissolvable (but impregnated, etc). However, for most practical applications the use of solid-walled hollow microneedles is often advantageous.

However, the production of such solid-walled hollow microneedles, which typically may have dimensions of only a few micrometers and may have specially defined external shapes, is not easy, especially since it is difficult to achieve microneedle structures of the required tiny sizes and wall thicknesses which have the required wall strengths and define cavity shapes that ensure optimum transfer of liquid active compositions to the recipient. This is reflected both in the various physical technical limitations of known microneedle arrays/patches/chips and also in the economics of their manufacture and thus their price in the marketplace.

It is a primary object of the present invention to address the above shortcomings and limitations of the known art of microneedles and provide a new approach and new techniques for the manufacture of microneedles, especially hollow microneedles, and especially which may enable the production of such microneedles with greater variability of specially designed profile shapes, different wall profiles and internal cavity shapes of individual microneedles, whilst maintaining low production costs.

### SUMMARY OF THE INVENTION

Accordingly, in a first aspect the present invention provides a method for the manufacture (or formation) of one or more microneedles, the method comprising:
(i) providing a substrate structure of electrically insulating material including one or more concavities extending into the body of the substrate structure from a face thereof, the one or more concavities being internally shaped and configured so as to correspond to the external shape and configuration of the respective one or more microneedles to be formed;
(ii) applying selectively to at least one first surface or surface portion of each of the said one or more concavities a layer of an electrically conductive material; and
(iii) using a galvanoplastic technique, depositing on each layer of electrically conductive material in the said one or more concavities at least one layer or body of microneedle wall-forming material so as build up a wall of a respective microneedle in each respective said concavity.

In many (and in practice probably in the majority of) embodiments of the invention, the method may be used to manufacture or form one or more hollow microneedles, i.e. microneedle(s) which each have a definable (and preferably single) channel or bore, of any suitable or desired longitudinal and/or transverse shape, extending internally therewithin from its base end to its opposite tip portion, for allowing passage therethrough of a liquid composition to be delivered by the or each respective microneedle upon use.

However, in certain other embodiments of the invention, the method may be designed for manufacturing or forming one or more non-hollow or substantially “solid” microneedles, i.e. microneedle(s) which each have a substantially solid body structure, optionally with open pores or voids (or even a plurality of micro/nano-channels) within its body structure and/or an outer coating/carrier surface, for transporting a liquid composition to be delivered by the or each respective microneedle upon use from its base end to its opposite tip portion via said pores/voids/channels and/or by virtue of passage over the said outer coating/carrier surface.

In many practical embodiments of the above-defined method of the first aspect of the invention, the method may include a preliminary step, prior to step (i), of providing a master substrate including one or more convexities protruding from a face thereof, the one or more convexities being externally shaped and configured so as to correspond to the desired or intended internal shape and configuration of the respective one or more concavities, and transferring the shape and configuration of the said convexities on the master substrate to the body of electrically insulating material of the said substrate structure so as to form the substrate structure that is then used in step (i) of the method.

In some embodiments of the invention, the application step (ii) may comprise applying selectively to the said at least one first surface or surface portion of each of the said one or more concavities a composition comprising or containing the said electrically conductive material or a precursor of the said electrically conductive material. Such a composition may be aqueous or comprise a solvent or dispersant which is substantially aqueous or hydrophilic.

For example, a reduced silver or a reduced palladium precursor composition may be used for this purpose. Such a precursor composition may typically be applied by spraying, and it may be provided for use in the form of a sprayable precursor composition comprising at least two components, which are chemically reactable together during or immediately after the application step (ii) (e.g. by a reduction process) to form the required layer of the said electrically conductive material. Practical examples of such precursor compositions based on silver or palladium may be well-known and already commercially available in the art.

In certain alternative embodiments, instead of forming the required layer of electrically conductive material in situ by spraying-on of a precursor composition therefor, the layer of electrically conductive material may alternatively be formed (e.g. in the case of an especially small-sized master substrate) by a vacuum deposition technique, in which a thin metal film or layer of the required conductive metal—e.g. Ag or Al—is applied by vacuum deposition (the typical practical details of which process are well-known in the art).

In some embodiments, if it is desired or deemed necessary, it may be possible for the application step (ii) to be preceded by a surface pre-treatment step, e.g. a chemical or physical surface pre-treatment step (e.g. a plasma pre-treatment), in which the surface(s) of the respective concavities are pre-treated either chemically or physically to facilitate the formation of the required layer of electrically conductive material thereon. For instance: for a precursor-composition-applied main electrically conductive material layer a suitable pre-treatment step may comprise the application of a pre-layer of Ag, Pt, or Ni, whereas for a vacuum deposition process for applying the main electrically conductive material layer a suitable pre-treatment step may comprise the application of a pre-layer of Al, Ag, Au or Cu. Other examples of metals for such surface pre-treatment steps may however be possible. In practice, in such embodiments that employ such a surface pre-treatment step, ultimately in such embodiment methods the pre-treatment layer may be removed from the substrate in a subsequent step of the overall method, once the microneedle walls have been formed.

In some embodiments of the above-defined method, the step (ii) may include or inherently/implicitly comprise, in addition to the applying selectively to the at least one first surface or surface portion of each of the said one or more concavities the layer of the electrically conductive material, an action or step or implicit/inherent effecting of not applying the said electrically conductive material to at least one second surface or surface portion of each of the said one or more concavities, wherein the said second surface(s) or surface portion(s) is/are different from the said first surface(s) or surface portion(s) of each concavity.

In some such embodiments as in the preceding paragraph, in order to achieve the said non-application of the electrically conductive material to the at least one second surface or surface portion of each concavity, the said at least one second surface or surface portion of each concavity may have been formed or pre-treated so as to include or present a surface which substantially prevents adherence or bonding thereto of the electrically conductive material or a composition used to deliver or apply same. Such a formed or pre-treated at least one second surface or surface portion of each concavity may comprise a textured surface or surface portion, such as a surface/surface portion which comprises an array or series or arrangement of a plurality of ridges, protrusions, protuberances, grooves, channels, indentations or other surface formations which act to substantially prevent adherence or bonding thereto of the electrically conductive material (or a composition used to deliver or apply same) which is applied to the concavities' first surface(s)/surface portion(s). In some such embodiments as above, the textured surface or surface portion of the at least one second surface or surface portion of each concavity may especially have or exhibit hydrophobic or superhydrophobic properties, particularly in the case where the composition from or via which the electrically conductive material is delivered or applied or formed is substantially aqueous or hydrophilic in nature.

In practising such embodiments as in the preceding paragraph, the provision of the textured at least one second surface or surface portion of each concavity (especially that which is/are hydrophobic or superhydrophobic in nature) may be effected by virtue of that/those second surface(s) or surface portion(s) having been formed by correspondingly externally shaped and configured portion(s) of the respective one or more convexities of a master substrate that is optionally used in a preliminary step as defined above for forming the substrate structure used in step (i) of the method by transferring to the body of electrically insulating material thereof the shape and configuration of the said convexities on the master substrate.

As an alternative to the above-mentioned physical means for providing or effecting such non-adherence or non-bonding of the electrically conductive material to the at least one second surface or surface portion of each concavity, in some alternative embodiments the same result may be achieved by virtue of the said at least one second surface or surface portion of each concavity in the substrate structure having been provided or pre-treated with a layer of a material which has or exhibits corresponding or equivalent hydrophobic or superhydrophobic properties (suitable practical examples of which are well-known in the art).

As a yet further alternative to the above-mentioned various means for providing or effecting the said non-adherence or non-bonding of the electrically conductive material to the at least one second surface or surface portion of each concavity, an alternative technique for ensuring that the said at least one second surface or surface portion of each concavity remain(s) electrically non-conductive even after the application step (ii) of the method, is for there to be included—either immediately before or immediately after or simultaneously with that step (ii)—an auxiliary method step of applying selectively to the said at least one second surface or surface portion of each of the said one or more concavities a layer of an electrically non-conductive or electrically insulating material (suitable examples of which are well-known in the art).

In some such embodiments as in the preceding five paragraphs, each said second surface or surface portion of each of the said one or more concavities may correspond to a respective tip portion of the respective microneedle to be formed in the said respective concavity, whereby each microneedle is formed with a tip portion including an open or hollow mouth or void or nozzle-like opening, i.e. where the microneedle walls have not been deposited by the galvanoplasty step (iii) selectively in that tip portion/region of the respective concavity of the substrate structure.

However, in some alternative embodiments to those in the preceding paragraph, each said second surface or surface portion of each said concavities may correspond to a respective sidewall portion of the respective microneedle to be formed in the said respective concavity, wherein the respective sidewall portion is a portion of a wall of the respective microneedle located between its tip portion and its opposite base end, whereby each microneedle is formed with a sidewall portion including an open or hollow mouth or void or opening, i.e. where the microneedle walls have not been deposited by the galvanoplasty step (iii) selectively in that sidewall portion/region of the respective concavity of the substrate structure.

For the purpose of carrying out an embodiment method as in the preceding paragraph, in which each microneedle is formed with an open or hollow mouth or void or opening in a sidewall portion thereof, rather than in a tip portion thereof, in a modified embodiment of the method of the present invention, the above-defined application step (ii) of the method of the first aspect of the invention may be modified so as to comprise the following sequence of sub-steps:
(iia) applying to at least one first surface of each of the said one or more concavities a layer of an electrically conductive material, especially wherein the at least one said first surface comprises substantially the whole of or at least a majority of the internal surface area of each concavity;
(iib) applying an electrically non-conductive optical resist layer over the applied layer of electrically conductive material on the said at least one first surface;
(iic) using a prefabricated mask with one or more predefined openings therein which allow passage therethrough of light of a wavelength designed to develop or cure or photoreact with the resist layer to anchor or secure or bond or unite it to or with the electrically conductive layer therebeneath selectively only in those one or more regions or portions or areas thereof which have been illuminated by the light having passed through the respective opening(s) in the mask; and
(iid) removing the material of the resist layer only in those one or more regions or portions or areas thereof which have not been so developed or cured or photoreacted by the above step (iic), so as to leave in place, anchored or secured or bonded or united to or with the electrically conductive layer therebeneath, one or more selected isolated remnant regions or portions or areas of the electrically non-conductive optical resist layer, whereby said one or more selectively formed remnant regions or portions or areas of the electrically non-conductive optical resist layer constitute the above-defined at least one second surface or surface portion of each of the said one or more concavities which has or exhibits electrically non-conductive properties and so does not permit galvanic electrodeposition thereon of the microneedle wall material in the subsequent galvanoplasty step (iii) of the method.

One or more practical examples of the above modified embodiment method will be described in further detail hereinbelow in the context of some currently preferred embodiments of the invention.

In many practical embodiments of the invention, the microneedle wall-forming material that is deposited in the galvanoplasty step (iii) may comprise a metal, e.g. nickel, or possibly any of various other metals as may be desired of the final microneedles and which lend themselves to being electro-depositable by a galvanoplastic technique. Examples of suitable such other metals will be readily recognisable and available to persons skilled in the art, and may include, for example, Cr, Cu and Ag. However, Ni may be especially preferred as the microneedles' wall material in many embodiments.

In many embodiments of the invention, in particular in those embodiments used to manufacture or form hollow microneedles, the microneedle wall-forming material that is deposited in the galvanoplasty step (iii) may be so applied or deposited in one or more discrete layer-forming steps so as to build up the microneedle walls so as to have an average overall wall thickness in the range of from about 0.01 up to about 1 mm.

However, in certain other embodiments which are designed to manufacture or form non-hollow or substantially “solid” microneedles, the galvanoplasty step (iii) may be designed so as to apply or deposit the appropriate microneedle wall-forming material (optionally with the above-defined pores/voids/channels formed inherently within its structure, if such are to be present) in a suitable number of layer-or body-forming steps so as to build up the or each respective substantially “solid” microneedle body structure from its outside towards its interior.

In many embodiments of the invention, each said first surface or surface portion of each of the said one or more concavities may comprise a major part of the respective concavity surface, especially which major part may extend circumferentially or peripherally substantially completely around the respective concavity, whereby that major part of the respective concavity surface corresponds to a respective major sidewall portion of the respective microneedle to be formed in that respective concavity.

In many practical embodiments of the above-defined method of the first aspect of the invention, the method may include a post-galvanoplasty step, subsequent to the depositing of the walls of the respective microneedles in step (iii), of removing the thus formed one or more microneedles from the substrate structure.

Furthermore, in some practical embodiments of the method of the invention, if appropriate or necessary the thus formed one or more microneedles may, once they have been removed from the substrate structure, be subjected to any suitable or desirable post-production step or treatment, such as laser cutting, e.g. to optimise exit hole sizes, remove excess or remnant unwanted wall material, trim the boundaries or edges of the microneedle array, patch or chip, etc.

Moreover, in some practical embodiments of the method of the invention, if appropriate or necessary the thus formed one or more microneedles may, once they have been removed from the substrate structure and been subjected to any appropriate physical post-production step or treatment, the outer walls of the microneedles may be subjected to a passivation or other surface-treatment step, in order to render them compatible with or safe to use on a recipient's or subject's (e.g. a patient's) skin. This may be highly desirable, or possibly even mandatory, for example in the case of certain metals (e.g. nickel) being used to form the microneedle walls and which metals may be inherently bio-incompatible or may exhibit a degree of toxicity that a recipient's/subject's or patient's skin needs to be shielded or protected from.

Suitable passivation techniques may include for example an “atomic layer deposition” (ALD) process, or other vapour deposition process. Generally, any passivating coating material may desirably be not only suitably biologically inert, but also it may desirably exhibit good adhesion to the metal or other material used to form the walls of the microneedles. Examples of suitable passivation coating materials and how to apply them, including so as not to compromise the bore widths or diameters of the microneedles themselves (especially at or in the vicinity of their tips) in order to maintain as far as possible their capability of allowing or promoting liquid flow thereacross or therepast, will be described in further detail hereinbelow in the context of some currently preferred embodiments of the invention.

In practising various embodiments of the method of the invention, the one or more concavities in the substrate structure (on whose at least one first surface or surface portion is applied the electrically conductive material layer) may be of any suitable or desired shape and configuration, especially any desired three-dimensional shape/configuration or transverse cross-section (relative to a longitudinal axis of the respective concavity substantially perpendicular to the direction in which the respective concavity extends into the body of the substrate structure material from its main outer face). For example, concavities with a generally cylindrical, or generally conical or frusto-conical, or alternatively a generally pyramidal, shape, with their transversely measured diameter or width decreasing passing in a direction away from the main outer face of the substrate structure, may often be used, in order to create, respectively, generally cylindrical or generally conical or generally pyramidal shaped microneedles.

Furthermore, the concavities may have sidewalls (or interior and/or exterior sidewall surfaces) which are of any desired or suitable simple or any of various more complex shapes/configurations, such as substantially smooth-sided, textured (or provided with surface formations), substantially straight-sided in a general longitudinal/axial direction, curved or arcuate (or wavy or irregularly or asymmetrically shaped) in a general longitudinal/axial direction, smoothly shaped or gently arcuate in generally transverse cross-section, wavy or irregularly or asymmetrically shaped in generally transverse cross-section, or even any of various other more complex shapes in three-dimensions.

In practising any of such embodiments as in the preceding paragraph, in the optional yet preferred case of using a master substrate in a preliminary step, prior to step (i) of the method, to predefine and transfer to the body of material forming the substrate structure (used in step (i)) the shape and configuration of the concavities therein, the said master substrate may be designed and formed accordingly, with its one or more convexities being externally shaped and configured so as to correspond to the desired or intended final internal shape and configuration of the respective one or more concavities of the resulting substrate structure. Thus, the master substrate itself that is used in the preliminary shape-transfer step may be likewise designed and shaped/configured with any desired or suitable simple or any of various more complex shapes/configurations as are desired for the resulting concavities of the substrate structure used in step (i) of the method.

Thus, in accordance with the general idea behind the present invention, it is based on the principle of using a substrate structure, which has been prepared (especially pre-prepared from an appropriately shaped and configured master substrate) with an indented or concavitied facial or surface profile which corresponds to the basic shapes of the one or more microneedles to be formed, as a former or “mould”, and modifying or treating selected sites or portions of its surface profile within each of its indents or concavities so as to exhibit selectively electrically conductive and (preferably) also selectively electrically non-conductive sites/portions, and then using a galvanoplasty technique to deposit or “grow” the respective microneedle walls selectively on only the electrically conductive sites/portions of the indents/concavities in the substrate structure. Furthermore, the desirable provision of appropriately positioned or located selectively electrically non-conductive sites/portions within each indent or concavity may allow more readily and assuredly the formation (especially in the case of hollow microneedles) of one or more required apertures or voids or (e.g. nozzle-like) openings in each respective microneedle once formed, e.g. so as to form a mouth or nozzle-like opening at its tip end or alternatively in a lateral sidewall thereof (e.g. if a mouth/nozzle/opening is required at such a location in the microneedles, rather than at their tips).

In many embodiments of the invention the method may be used to manufacture or form a plurality of microneedles substantially simultaneously in the form of an array, patch or chip comprising said plurality of microneedles protruding from a major face thereof. In many such embodiments the longitudinal axes of the plurality of microneedles may all be substantially parallel with one another, whereby all the microneedles point in substantially the same direction from the said major face of the array, patch or chip.

In such embodiments in which a plurality of microneedles are manufactured/formed, therefore, the substrate structure may include a plurality of the said concavities and the application step (ii) may comprise applying to at least one first surface or surface portion of each respective one of the plurality of concavities a respective said layer of the electrically conductive material, so that in the galvanoplastic deposition step (iii) a respective (at least one) layer or body of the microneedle wall-forming material is deposited on each one of the respective layers of electrically conductive material on the respective concavities' first surfaces or surface portions, so as to build up respective walls of the respective microneedles in the respective concavities.

In some such embodiments in which a plurality of microneedles are manufactured/formed as an array, patch or chip thereof, the plurality of microneedles may be joined together at their respective base ends by a unifying base or root or bridging portion extending commonly between all the said microneedles of the plurality. In an embodiment method of forming such an array, patch or chip of microneedles, in the application step (ii) the at least one first surface or surface of each of the said one or more concavities, on which is/are applied the said respective layers of electrically conductive material, may extend across defining boundaries between adjacent concavities and/or may extend from one concavity into an adjacent concavity.

In some such embodiment microneedle arrays or chips within the scope of the invention, in which the plurality of microneedles are joined together at their respective base ends by respective unifying bridging base portions extending between adjacent microneedles, it may be that it is desirable or necessary to produce such an array or chip with the bridging base portions having a greater thickness and thus strength as compared with the thickness of the walls of the microneedles themselves, in order to give the array/chip greater overall strength and rigidity and thus improved handling capability and resistance to damage etc during use. Thus, it may be that in certain embodiment methods within the scope of the invention a plural-stage galvanoplasty step (iii) may be carried out, wherein at least one first galvanoplasty stage is carried out so as to deposit at least one first layer or body of microneedle wall-forming material in order to form at least the walls of the microneedles and the respective bridging base portions between them, and subsequent thereto at least one second, but selective, galvanoplasty stage is then carried out to selectively deposit on the said respective bridging base portions only at least one second layer or body of microneedle wall-forming material in order to increase the thickness of those respective bridging base portions as compared with the thickness of the microneedle walls. The selective deposition of the said at least one second layer or body of microneedle wall-forming material on the said respective bridging base portions only may be achieved by application, after the first galvanoplasty stage, to those portions of the growing microneedle array or chip other than the bridging base portions—i.e. preferably onto at least the microneedles' wall portions themselves—a temporary coating or filler or masking layer of an electrically non-conductive material, e.g. a wax or plastics material, which substantially prevents any further build-up of the microneedles' wall thickness in those wall portions themselves, but only allows such build-up of the wall thickness in the bridging base portions only during the at least one second galvanoplasty stage. In this manner, using a second or subsequent, but selective, galvanoplasty stage during the overall galvanoplasty step (iii) of the method to selectively build up the base region only of the microneedle array or chip to a desired increased thickness and thus strength, the base of the final resulting microneedle array or chip may be suitably and selectively strengthened. After completion of the second galvanoplasty stage, the non-conductive wax or other coating/filler/masking layer may be simply removed, e.g. by dissolution using a suitable solvent.

In practising various embodiments of the invention, by suitably adjusting one or more parameters of the galvanoplasty step (iii)—such as the extent of any shielding (i.e. using a shield plate of a suitable specific shape placed between the layer to be plated and the second electrode in the galvanic bath), the strengths of the electric or magnetic fields used, changes in current values over time, etc—it may be possible to create microneedles with particular or specially designed shapes or wall properties, e.g. by building up each microneedle's wall through the deposition of one or more layers or bodies thereof so that the resulting microneedle adopts a particular desired profile or three-dimensional shape, or possibly so that its wall(s) has/have or exhibit(s) a particular desired degree of hardness or toughness (or other physical property or attribute), as may be desired of the finally produced microneedles. Such bespoke tailoring of the resulting microneedles' shapes, properties or other attributes etc may be accomplished by designing the overall conditions and parameters of the galvanoplasty electrodeposition step (iii) appropriately, especially by suitable modelling.

As another advantage of the present invention, it may be mentioned that because of the use of the galvanoplasty electrodeposition step (iii) to in effect “grow” the respective microneedle walls selectively on only the electrically conductive sites/portions of the indents/concavities in the substrate structure, once the microneedles have been so “grown” and removed from the substrate structure, the substrate structure may be reused for the production of another one or more (especially another array, patch or chip of) microneedles. This reusability of the substrate structure may therefore improve the overall economics of manufacture of large numbers of such microneedle arrays, patches or chips.

In a second aspect the present invention provides a substrate structure for use in a method of the first aspect of the invention or any embodiment thereof, the substrate structure comprising a body of electrically insulating material including one or more concavities extending into the body thereof from a face thereof, and the one or more concavities being internally shaped and configured so as to correspond to the external shape and configuration of the respective one or more microneedles to be formed in the said method, wherein at least one first surface or surface portion of each of the said one or more concavities has applied thereto a layer of an electrically conductive material.

In some embodiments of the above-defined substrate structure of the second aspect, each of the said one or more concavities may include at least one second surface or surface portion thereof which is/are different from the said first surface(s) or surface portion(s) thereof, and each said second surface or surface portion of each concavity may have been formed or pre-treated so as to include or present a surface which substantially prevents adherence or bonding thereto of the electrically conductive material (or a composition used to deliver or apply or form same) during the application step (ii) of the method. In some such embodiments, each said second surface or surface portion of each concavity may comprise a textured surface or surface portion, such as a surface/surface portion which comprises an array or series or arrangement of a plurality of ridges, protrusions, protuberances, grooves, channels, indentations or other surface formations which act to substantially prevent adherence or bonding thereto of the electrically conductive material (or a composition used to deliver or apply or form same) during the application step (ii) of the method. In some such embodiments, the textured surface or surface portion of the at least one second surface or surface portion of each concavity may especially have or exhibit hydrophobic or superhydrophobic properties, particularly in the case where a composition from or via which the electrically conductive material is to be delivered or applied or formed is substantially aqueous or hydrophilic in nature. In some alternative embodiments, as an alternative to the aforementioned physical means for providing or effecting such non-adherence or non-bonding of the electrically conductive material to the at least one second surface or surface portion of each concavity, the said at least one second surface or surface portion of each concavity in the substrate structure of this aspect may instead have been provided or pre-treated with a layer of a material which has or exhibits corresponding or equivalent hydrophobic or superhydrophobic properties (suitable practical examples of which are well-known in the art).

In some embodiments of the substrate structure of the preceding paragraph, each said second surface or surface portion of each concavity may correspond to a respective tip portion of the respective microneedle to be formed in the said respective concavity, whereby each microneedle is formable in the method with a tip portion including an open or hollow mouth or void or nozzle-like opening, i.e. where the microneedle walls have not been deposited by the galvanoplasty step (iii) in that portion/region of the respective concavity of the substrate structure. However, in some alternative embodiments to these, each said second surface or surface portion of each concavity may correspond to a respective sidewall portion of the respective microneedle to be formed in the said respective concavity, wherein the respective sidewall portion is a portion of a wall of the respective microneedle located between its tip portion and its opposite base end, whereby each microneedle is formable in the method with a sidewall portion including an open or hollow mouth or void or opening, i.e. where the microneedle walls have not been deposited by the galvanoplasty step (iii) in that portion/region of the respective concavity of the substrate structure.

In a third aspect the present invention provides one or more microneedles, especially one or more hollow microneedles, and especially an array, patch or chip comprising same, the one or more said microneedles being formed by a method according to the first aspect of the invention or any embodiment thereof. Within the scope of this third aspect of the invention is the provision of a plurality of microneedles, especially a plurality of hollow microneedles, and especially in the form of an array, patch or chip comprising same, the said plurality of microneedles being formed substantially simultaneously with each other by a method according to the first aspect of the invention or any embodiment thereof applicable to the case of it being used to form a plurality of microneedles.

Embodiments of the invention in its various aspects may be applicable to the manufacture or formation of microneedles of a wide variety of sizes and shapes and configurations, as already mentioned. For instance, the microneedles produced by use of embodiments of the invention may typically have sizes in the approximate ranges of around 150 to around 1500 μm in longitudinal (i.e. axial) length, around 50 to around 500 μm in transverse width or diameter, and with open/hollow mouth or void or nozzle-like openings around 1 to around 100 μm in width or diameter.

For the purpose of practising various embodiments of the invention in its various aspects, suitable examples of standard processing steps, techniques, apparatuses and process parameters that may be used for effecting the various steps of the manufacturing methods will be well-known in the art and within the everyday knowledge and skill of those working in the microneedles art. Furthermore, various specific examples of many such features, as well as examples of typical or preferred parameters or parameter ranges used in their practical application and utilisation, will be apparent from the specific descriptions of some currently preferred embodiments which follow hereinbelow.

Within the scope of this specification it is envisaged that the various aspects, embodiments, examples, features and alternatives, and in particular the individual constructional, configurational or operational features thereof, set out in the preceding paragraphs, in the claims and/or in the following description and accompanying drawings, may be taken independently or in any combination of any number of same. For example, individual features described in connection with one particular embodiment, or described singly or in combination with another feature in any one or more embodiments, are applicable on their own or in combination with one or more other features to all embodiments and may be found and used in combination with any other feature in any given embodiment, unless expressly stated otherwise or such features are incompatible.

### BRIEF DESCRIPTION OF THE DRAWINGS

Some embodiments of the present invention in its various aspects will now be described in detail, by way of non-limiting example only, with reference to the accompanying drawings, in which:

FIG. 1 is a schematic sectional view, considerably enlarged, of an example of a master substrate that is used at the outset in a method of manufacturing a microneedles array, patch or chip in accordance with a first embodiment of the invention;

FIG. 2 is a schematic sectional view, considerably enlarged, of an example of a corresponding replicated substrate structure which has been subjected to a first preparative step in the manufacturing method according to the invention embodiment, and is ready for the “growing” of the microneedles thereon;

FIG. 3 is a schematic sectional view, considerably enlarged, of an example of the finally “grown” microneedles formed on the prepared substrate structure of FIG. 2;

FIG. 4 is a schematic sectional view, considerably enlarged, of a prepared substrate structure that is used in a manufacturing method according to a second embodiment of the invention, in which masking is used to create specially positioned output holes in the individual microneedles;

FIG. 5 is a schematic sectional view, considerably enlarged, of the prepared substrate structure of FIG. 4 showing it undergoing a UV exposure step as part of the second embodiment manufacturing method;

FIG. 6 is a schematic sectional view, considerably enlarged, of the “developed” substrate structure of FIG. 5, showing it ready for the “growing” of the microneedles thereon in accordance with this overall second embodiment manufacturing method;

FIG. 7 is a schematic sectional view, considerably enlarged, of the finally “grown” microneedles, with specially positioned output holes, formed on the prepared substrate structure of FIG. 6 in accordance with this overall second embodiment manufacturing method.

### DETAILED DESCRIPTION OF EMBODIMENTS

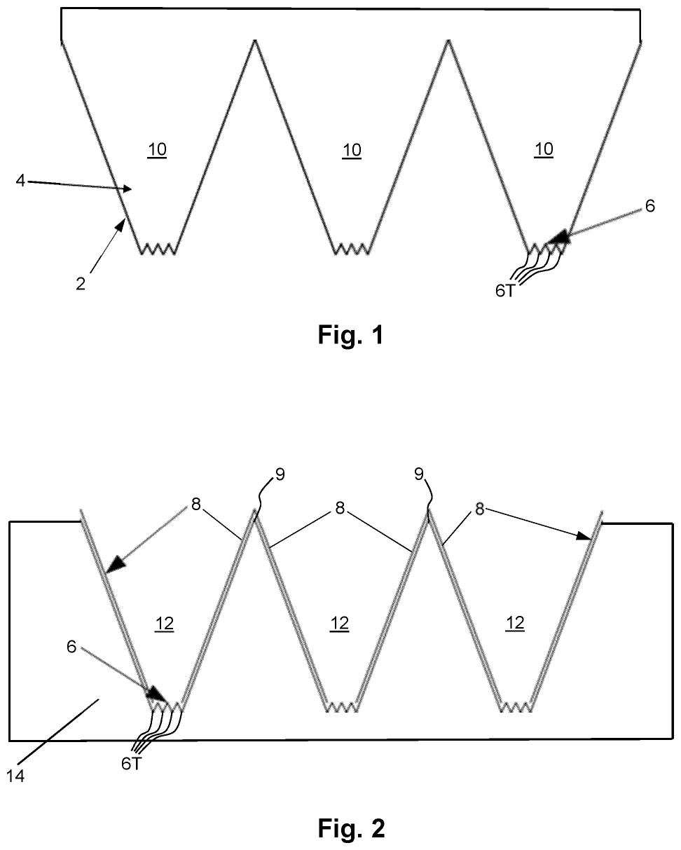

Referring firstly to FIGS. 1 to 3, these FIGS. illustrate the main key production steps used in a first embodiment of the manufacturing method according to the present invention, and show, respectively, the initial master substrate used to prepare the substrate structure on which hollow microneedles are “grown” by galvanoplastic electrodeposition, the replicated substrate structure itself which has been prepared ready for carrying out the galvanoplastic electrodeposition step by the application to selected surface portions of the interiors of the concavities in the substrate structure of an electrically conductive material, and the substrate structure after the galvanoplastic electrodeposition step has been carried out thereon and the hollow microneedles' walls formed or “grown” on the treated selected surface portions of the concavities.

These main production steps are, in further detail, as follows:

### Production Step 1

As shown in FIG. 1, the first, i.e. preliminary, step of the overall manufacturing method according to this first invention embodiment begins with the production of a master substrate shown generally as 2, comprising a unitary body 4 of a suitable non-metallic material, especially a polymer or glass or ceramic-based material. Examples of suitable master substrate body materials include: polymers, such as PET (polyethylene terephthalate), PC (polycarbonate) and PMMA (poly (methyl methacrylate)); glasses; silicon (Si); silicones; Ormocers (“Organically Modified Ceramics”, i.e. a family of three-dimensionally cross-linked copolymers comprising organic and inorganic components as well as a polysiloxane component); photopolymers; SU8 (an epoxy-based photoresist); as well as others. Specific practical examples of the aforementioned master substrate body materials are well-known and widely commercially available.

This master substrate 2 comprises a series or array of convex conical protrusions 10 which define the resulting shape and configuration of the various microneedles—or rather the resulting exterior shape and configuration of the walls of the various microneedles—that are to be produced. The purpose of the shaped master substrate 2 is to transfer the individual shapes and configurations, as well as the overall pattern/layout/arrangement, of the various convex conical protrusions 10 to the body of electrically insulating material that forms the replicated substrate structure 14 (FIG. 2) to form the required concavities 12 therein, which concavities 12 are then used to “grow” the microneedles therein in the later galvanoplasty step of the overall method.

In the master substrate 2, at the lower, narrowest end of each convex conical protrusion 10, its end face is formed with, or so as to present, a textured end surface 6 comprising a series of closely-spaced ridges, protrusions, protuberances, grooves, channels, indentations or other surface formations 6T which, when their shapes are transferred from the master substrate 2 to the bottom walls of the concavities 12 formed in the body of electrically insulating material forming the main substrate structure 14 (FIG. 2) used in step (i) of the method, impart to those bottom walls of the concavities 12 corresponding textured end surfaces 6 (with corresponding surface formations 6T), which thereby have or exhibit hydrophobic or superhydrophobic properties. This is so as to substantially prevent adhesion or bonding to those lower end walls of the concavities 12 of an aqueous composition used in step (ii) to apply to the interior sidewalls of the concavities 12 the layer of electrically conductive material on which the microneedles' walls are then “grown” in the galvanoplasty step (iii).

The complex surface structure of the master substrate 2, with its series or array of convex conical protrusions 10 and their textured end surfaces 6 may be formed by any suitable known method or technique for the production of such micron-scale complex shaped surfaces in the arts of nano-(or micro-) imprint lithography and nano-(or micro-) 3D printing technology. For example, layer-by-layer additive manufacturing using femtosecond laser pulses to polymerize photosensitive material may be one known technique that may be used. This technique is often known as “two photon polymerization” and allows the making of structures on a sub-micron scale of size. Other examples of other known techniques may of course also be available and be used instead.

### Production Step 2

As per step (i) of the method of the invention, the master substrate 2 of FIG. 1 is then used to form by replication the main substrate structure 14 which defines the arrangement, layout and individual complex shapes of the various concavities 12 on whose interior sidewalls are to be “grown” the microneedles' walls in the subsequent galvanoplasty step (iii). The substrate structure 14 is formed of a body of electrically insulating (i.e. substantially non-conductive) material, such as various known insulating materials exemplified by any of the example materials mentioned above for making the master substrate—that is to say, suitable examples of the material used to form the main substrate (14) may include: [A1] plastics or [A2] [A3] polymers, such as PET (polyethylene terephthalate), PC (polycarbonate) and PMMA (poly (methyl methacrylate)); glasses; Ormocers (“Organically Modified Ceramics”, i.e. a family of three-dimensionally cross-linked copolymers comprising organic and inorganic components as well as a polysiloxane component); photopolymers; SU8 (an epoxy-based photoresist); as well as others. Specific practical examples of all these materials are well-known and widely commercially available.

The complex surface structural shape of the master substrate 2 is transferred to a “blank” body of the electrically insulating material to form the main substrate structure 14 by any suitable known technique used in the art of micro-or nano-scale printing, imprinting, stamping or embossing, practical examples of which are well-known in those arts. For example, a heat embossing technique may typically be used for this purpose. Other suitable techniques usable for this purpose may include nano-imprinting (e.g. in which the surface relief shape is copied into a soft layer from a UV-sensitive lacquer that is fixed by UV radiation or copied into a soft layer that is fixed by a chemical reaction (e.g. using silicones, that may be heated or not)). Yet other possible example techniques may of course also be available and be used instead, and specific practical details of all such known techniques usable for this purpose will be well-known and widely practised by people skilled in the art.

### Production Step 3

As shown in FIG. 2, the prepared main substrate structure 14 is then treated, as per step (ii) of the method of the invention, so as to apply to selected portions only of the interior walls of each of the various concavities 12 therein a thin layer 8 (e.g. up to around 100 nm in thickness) of an electrically conductive material, which selectively applied conductive layers 8 are then used to “grow” thereon the microneedles' walls in the subsequent galvanoplasty step (iii) of the method.

The electrically conductive layer material 8 may preferably be applied in the form of a precursor composition, especially an aqueous precursor composition, e.g. a reduced silver or a reduced palladium precursor composition. Such a precursor composition may be provided for use in the form of a sprayable liquid precursor composition comprising at least two components which are chemically reactable together during or immediately after the application step (e.g. by a reduction process) to form the required layer of the electrically conductive material. Practical examples of such precursor compositions based on silver or palladium are well-known and commercially available in the art. The precursor composition may be applied by any suitable practical technique, e.g. spraying, using any suitable known spraying apparatus.

(However, in certain modified, alternative embodiments, instead of forming the layer 8 of electrically conductive material in situ by spraying-on of a precursor composition therefor, that layer 8 of electrically conductive material may instead be formed (e.g. in the case of an especially small-sized master substrate) by a vacuum deposition technique, in which a thin metal film or layer of the required conductive metal—e.g. Ag or Al—is applied by vacuum deposition (the typical practical details of which process are well-known in the art). Furthermore, if it is desired or deemed necessary, it may be possible for the application step (ii) to be preceded by a surface pre-treatment step, e.g. a chemical or physical surface pre-treatment step (e.g. a plasma pre-treatment), in which the surface(s) of the respective concavities 12 are pre-treated either chemically or physically to facilitate the formation of the required layer 8 of electrically conductive material thereon. For instance: for a precursor-composition-applied main electrically conductive material layer a suitable pre-treatment step may comprise the application of a pre-layer of Ag, Pt, or Ni, whereas for a vacuum deposition process for applying the main electrically conductive material layer a suitable pre-treatment step may comprise the application of a pre-layer of Al, Ag, Au or Cu. Other examples of metals for such surface pre-treatment steps may however be possible.)

Because of the inherent hydrophobic (or superhydrophobic) property of the textured bottom walls 6 of the various concavities 12 in the substrate structure 14, the aqueous precursor composition applied to the internal walls of the concavities 12 to form the electrically conductive layers 8 thereon substantially does not adhere or bond to those lower end walls 6, and therefore those lower end walls 6 remain uncoated with and free from the precursor composition and thereby end up free from and uncoated with the resulting electrically conductive material. This therefore leads to those end wall portions of the various concavities not having grown thereon any wall material which otherwise forms the microneedles' walls during the subsequent galvanoplasty step (iii) of the method.

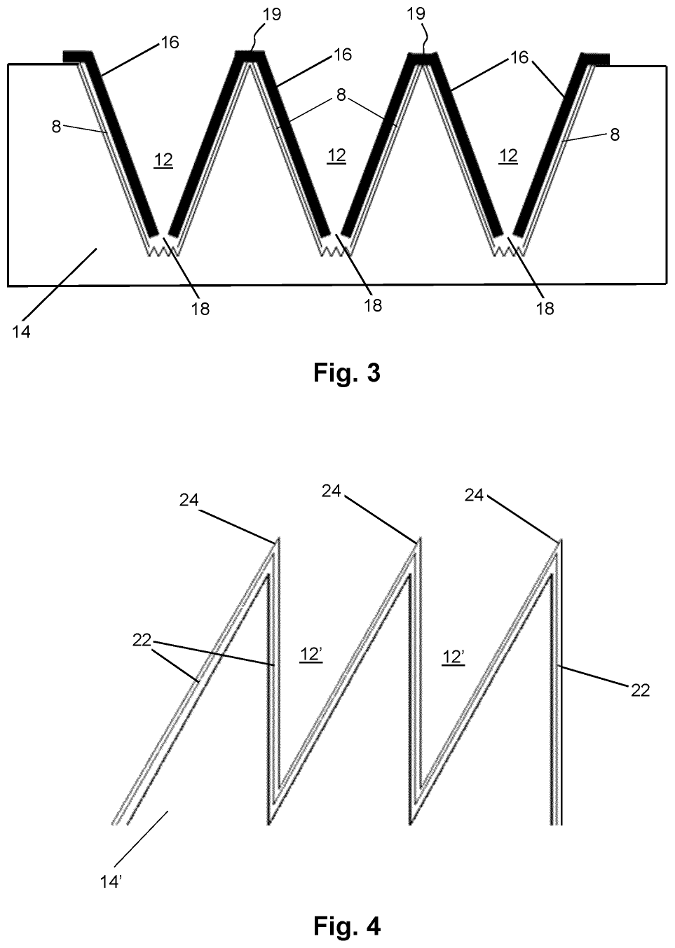

It will be noted also that the applied layers 8 of electrically conductive material extend right across the boundaries 9 between adjacent concavities 12, i.e. from the interior of one concavity 12 into the adjacent one, in order that the subsequently “grown” microneedles' walls likewise extend across the defining boundaries between adjacent microneedles and thus extend from one microneedle's interior into an adjacent microneedle's interior, which arrangement can therefore form a unified or unitary array, patch or chip of the resulting microneedles in which the individual microneedles are united at their base (i.e. upper) ends e.g. by respective bridging portions as at 19 in FIG. 3.

### Production Step 4

As shown in FIG. 3, and as per step (iii) of the method of the invention, the substrate structure 14 which has thus been partially and selectively coated in the interiors of its concavities 12 with the layers of electrically conductive material 8 is then subjected to a galvanoplasty process in which each of the electrically conducive layers 8 is galvanically plated with one or more layers of a metal which is to form the actual walls 16 of each microneedle. Suitable metals for this wall-forming galvanic electrodeposition process include nickel, although other metals may be suitable and used instead, with appropriate adjustment of the galvanic constituents and conditions employed for the electrodeposition. Examples of suitable such other metals will be readily recognisable and available to persons skilled in the art, and may include, for example, Cr, Cu and Ag. However, Ni may be especially preferred as the microneedles' wall material in many practical embodiments. Examples of particular galvanic apparatus and precise constituents and process parameters and conditions that are employable for the electrodeposition process will be readily understood, available and already practised by people skilled in the art of known galvanoplasty techniques that are already well-known and widely practised in the art, so do not require any further specific elucidation here.

The final thickness of the hollow microneedles' walls 16 may be dictated or controlled for example by the conditions, time duration and possibly other parameters of the galvanoplastic electrodeposition process of this step (iii) of the method. Typical hollow microneedle wall thicknesses which are galvanically deposited by means of this process may for example be in the range of average overall wall thicknesses in the region of from about 0.01 up to about 1 mm (i.e. from about 10 to about 1000 μm).

It may further be possible, by suitable control of the various conditions and parameters of the galvanoplastic electrodeposition process, for the hollow microneedles' wall thickness to vary passing along each microneedle in its longitudinal or axial direction, or even in one or more other directions along or across it. For example, it may be desirable to form the microneedle walls with a decreasing thickness passing towards their tip ends, in order to create a sharper and more pointed tip of each microneedle.

Moreover, if need be or if it is appropriate, instead of a single galvanic deposition step being used to “grow” the required microneedle walls 16 on the applied electrically conductive layers 8, a plurality of sequential discrete galvanic deposition sub-steps may be used instead to build up a plurality of discrete microneedle wall layers, one on top of another, so as to build up the hollow microneedles' walls 16 to a desired total thickness.

Furthermore still, it may be possible, by suitable selection and control of the conditions, time duration and possibly other parameters of the galvanoplastic electrodeposition process, to “grow” the hollow microneedle walls 16 such that they have specially designed three-dimensional shapes or configurations, e.g. in a case where complex-shaped microneedles may be required.

Furthermore yet still, in an alternative, modified, embodiment for the formation of non-hollow or substantially “solid” microneedles (which are not illustrated in the drawings, but are made using the same principles), it may be possible, again by suitable selection and control of the materials, conditions, time duration and possibly other parameters of the galvanoplastic electrodeposition process, to “grow” the microneedle walls in a stepwise or even continuous process from the outside inwards so as to build up substantially “solid” microneedle body structures that substantially completely fill the transverse widths of the respective concavities 12.

As shown in FIG. 3, owing to the non-coated lower end walls 6 in each concavity 12 (as a result of their hydrophobicity or superhydrophobicity during the application step (ii) of the method), no deposition by electroplating of the metal microneedle wall material occurs on those end wall portions of the concavities 12, which thereby creates a hole 18 in each of those microneedle end walls of each formed hollow microneedle 16—which holes 18 thus each constitute an open or hollow mouth or nozzle-like opening in the tip end of each microneedle 16 via which a liquid product can be injected through each microneedle 16 during their subsequent practical use.

Once the plurality of microneedles have been appropriately “grown” on the selectively coated concavities of the substrate structure 14 (as shown in FIG. 3)—to thereby form an array, patch or chip of those microneedles—they can then be removed from the substrate structure 14 by simple mechanical removal, optionally using a tool to assist their handling. Preferably this removal may be accomplished by removing the complete array, patch or chip of the plurality of microneedles 16 as a single entity, with the various microneedles united into a unitary array, patch-or chip-like arrangement at their upper base ends (as at respective bridging base portions 19 in FIG. 3) as a result of the continuities of the applied electrically conductive layers 8 extending between adjacent concavities 12 (as in FIG. 2).

Thereafter, if it should be necessary or appropriate, once the thus formed microneedle array, with the microneedles united thereinto via the various bridging base portions 19, has been removed from the substrate structure 14, it may be subjected to any appropriate or desirable post-production step or treatment, such as laser cutting, e.g. to optimise exit hole sizes, remove excess or remnant unwanted wall material, trim the boundaries or edges of the microneedle array, patch or chip, etc.

In some embodiment microneedle arrays or chips, including those made as shown in FIG. 3, it may be that it is desired or necessary to produce such an array or chip with the bridging base portions 19 having a greater thickness and thus strength as compared with the thickness of the walls 16 of the microneedles themselves, in order to give the array/chip greater overall strength and rigidity and thus improved handling capability and resistance to damage etc during use. Thus, it may be that in certain embodiment methods within the scope of the invention a plural-stage galvanoplasty step (iii) is carried out, wherein at least one first galvanoplasty stage is carried out so as to deposit at least one first layer or body of microneedle wall-forming material in order to form at least the walls 16 of the microneedles and the respective bridging base portions 19 between them, and subsequent thereto at least one second, but selective, galvanoplasty stage is then carried out to selectively deposit on the said respective bridging base portions 19 only at least one second layer or body of microneedle wall-forming material in order to increase the thickness of those respective bridging base portions 19 as compared with the thickness of the microneedle walls 16. The selective deposition of the said at least one second layer or body of microneedle wall-forming material on the said respective bridging base portions 19 only may be achieved by application, after the first galvanoplasty stage, to those portions of the growing microneedle array or chip other than the bridging base portions 19—i.e. preferably onto at least the microneedles' wall portions 16 themselves—a temporary coating or filler or masking layer of an electrically non-conductive material, e.g. a wax or plastics material, which substantially prevents any further build-up of the microneedles' wall thickness in those wall portions 16 themselves, but only allows such build-up of the wall thickness in the bridging base portions 19 only during the at least one second galvanoplasty stage. In this manner, using a second or subsequent, but selective, galvanoplasty stage during the overall galvanoplasty step (iii) of the method to selectively build up the base region only of the microneedle array or chip to a desired increased thickness and thus strength, the base of the final resulting microneedle array or chip can be suitably and selectively strengthened. After completion of the second galvanoplasty stage, the non-conductive wax or other coating/filler/masking layer may be simply removed, e.g. by dissolution using a suitable solvent.

### Production Step 5

Following the “growing” of the collection of microneedles 16 and their removal from the substrate structure 14 (as in FIG. 3), if desired or necessary the outer walls of the microneedles may be subjected to a passivation or other surface-treatment step, in order to render them compatible with or safe to use on a recipient's or subject's (e.g. a patient's) skin. This may be highly desirable, or possibly even mandatory, for example in the case of certain metals (e.g. nickel) being used to form the microneedle walls and which metals may be inherently bio-incompatible or may exhibit a degree of toxicity that a recipient's/subject's or patient's skin needs to be shielded or protected from.

Generally it may be important that the passivation coating material is not only suitably inert to the human body, but it also exhibits good adhesion to the metal or other material used to form the walls of the microneedles. Examples of suitable passivation coating materials for use in the context of embodiments of this invention may include: titanium nitride, aluminium oxide, silicon, as well as possibly others.

Several techniques can be used for such a passivation step to render the outer wall surfaces of the individual microneedles inert to the human body for this purpose, such as chemical vapour deposition processes, e.g. an “atomic layer deposition” (ALD) process (which involves thin-film deposition based on the sequential use of a gas-phase chemical process). Other passivation processes or techniques may also be suitable. Such ALD or other surface deposition techniques are per se well-known in the art, as are the apparatuses and procedural steps used to effect them.

Generally speaking any ALD or other surface deposition technique for the purpose of passivation of the outer wall surfaces of the microneedles may desirably be effected so as not to deleteriously reduce the size of, or detract from the surface properties of, the internal bore widths or diameters of the microneedles (especially at or in the vicinity of their tips), in order to maintain as far as possible their capability of allowing or promoting liquid flow thereacross or therepast, especially when a liquid composition is to be delivered to a subject or recipient via the finally manufactured microneedle array, patch or chip.

Having described in some detail many of the key production steps involved in practising many embodiments of the manufacturing methods according to the present invention, various optional modifications or additional features may be made or included in other embodiments in order to tailor such other embodiment methods better to the production of microneedles or microneedle arrays/patches/chips for various specific end applications or microneedle constructions/configurations for particular end uses. Some of these further options are described as follows:

### Optional Masking of Surfaces of Substrate Structure

The example embodiments described above in relation to FIGS. 1 to 3 all involve the manufacture or formation of hollow microneedles 16 of the type which have an open or hollow mouth or nozzle-like opening 18 at their tip ends (as in FIG. 3). However, by suitable modification of the method and the main substrate structure used to define the nature and positioning of the sites in each concavity thereof on which the electrically conductive material layers are formed for the “growing” thereon of the microneedle wall material, it may be possible to design embodiment methods and resulting embodiment microneedles in which the open/hollow mouths or voids or nozzle-like openings in each microneedle are positioned or located not a tip thereof but in a sidewall portion thereof, e.g. intermediate its tip portion and opposite base end or at a suitable distance longitudinally along the sidewall thereof somewhere between its tip and opposite base ends.

In this case the application step (ii) of the broadly defined method of the invention may be modified so as to comprise the following sequence of sub-steps:
(iia) applying to at least one first surface of each of the one or more concavities a layer of an electrically conductive material, especially wherein the at least one first surface comprises substantially the whole of or at least a majority of the internal surface area of each concavity;
(iib) applying an electrically non-conductive optical resist layer (e.g. a UV resist layer) over the applied layer of electrically conductive material on the at least one first surface;
(iic) using a prefabricated mask with one or more predefined openings therein which allow passage therethrough of light (e.g. UV) of a wavelength designed to develop or cure or photoreact with the resist layer to anchor or secure or bond or unite it to or with the electrically conductive layer therebeneath selectively only in those one or more regions or portions or areas thereof which have been illuminated by the light having passed through the respective opening(s) in the mask; and
(iid) removing the material of the resist layer only in those one or more regions or portions or areas thereof which have not been so developed or cured or photoreacted by the above step (iic), so as to leave in place, anchored or secured or bonded or united to or with the electrically conductive layer therebeneath, one or more selected isolated remnant regions or portions or areas of the electrically non-conductive optical resist layer, whereby the one or more selectively formed remnant regions or portions or areas of the electrically non-conductive optical resist layer constitute the above-defined at least one second surface or surface portion of each of the one or more concavities which has or exhibits electrically non-conductive properties and so does not permit galvanic electrodeposition thereon of the microneedle wall material in the subsequent galvanoplasty step (iii) of the method.

Thus, in the above modified method step (ii), the locations or positioning of the predefined openings in the prefabricated mask may be chosen or arranged appropriately, relative to the substrate structure with the electrically non-conductive optical resist layer applied over the already applied layer of electrically conductive material on the at least one first surface of each of the one or more concavities, such that the light transmitted through the mask openings creates, generates or forms, following removal of the resist layer on those non-illuminated regions/portions/areas thereof, the remnant regions or portions or areas of the electrically non-conductive optical resist layer selectively in the precisely desired microneedle-sidewall-corresponding locations thereon and with the precisely desired dimensions and shapes, ready for defining those precisely designed one or more open mouths or voids or nozzle-like openings in each microneedle sidewall upon the “growing” by the galvanoplastic electrodeposition of the relevant microneedle sidewall material only on the remaining electrically conductive sidewall portions thereof in the subsequent deposition step (iii) of the method.

The various main stages in the above modified method are illustrated sequentially in FIGS. 4, 5, 6 and 7.

As shown in FIG. 4, an electrically conductive layer 22 (like that conductive layer 8 in FIG. 2) is coated on the entire internal surfaces of the concavities 12′ of the main substrate structure 14′, and a UV photoresist layer 24 is then applied over this electrically conductive layer 22. Suitable UV photoresist materials include various examples thereof that are well-known in the art. For instance, various negative or positive tone photoresists based on various chemistries may typically be used: such as DNQ (diazonaphthoquinone), non-DNQ (e.g. chemically amplified resists), epoxy-based or acrylic monomers-based resist types. For example, commercially available negative tone photoresist materials include: SU-8 2000series resists; AZ15nXT; AZ 125nXT; AZ nLOF 2000 Series resists; AR-N 4400 series resists; AR-N 2200 series resists; as well as others. Examples of commercially available positive tone photoresist materials include: AZ 1500 series resists; ma-P 1200 series resists; AR-P 3000 series resists; AR-P 5300 series resists. (Note: Novolac resins, on which some of the above example resists are based, are a family of phenolic polymer based resins, comprising low molecular weight polymers derived from phenols and formaldehyde, and are well-known in the art and widely commercially available.)

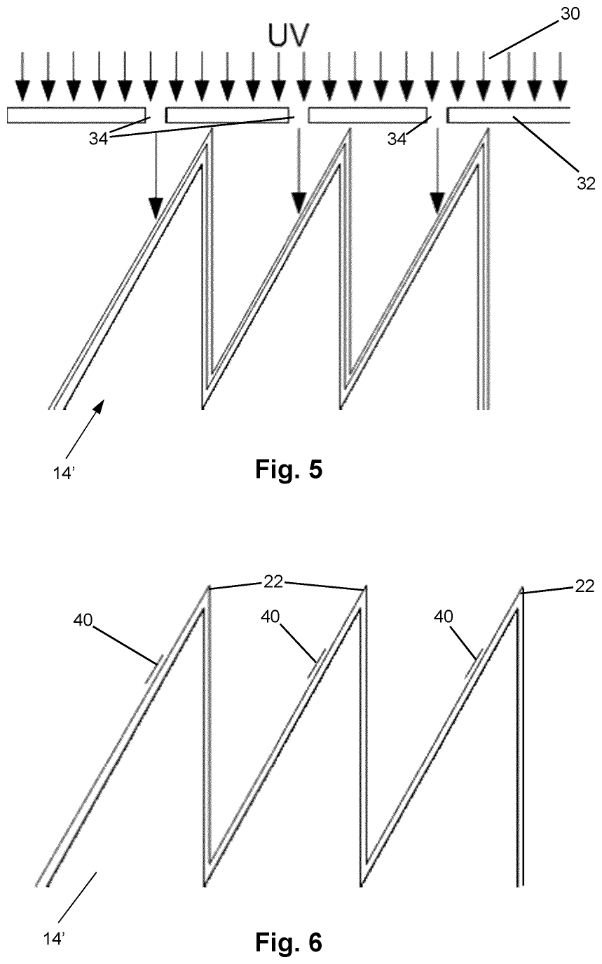

Then, as shown in FIG. 5, a prefabricated mask 32 of UV-opaque material (various specific examples of which are well-known in the art and widely commercially available) with appropriately dimensioned and positioned apertures or holes 34 therein is laid over the substrate structure 14′, and the combined arrangement is then illuminated with UV (ultraviolet) light 30, e.g. from a collimated UV source (not shown), so that only those selected areas or portions of the resist layer 24 which are illuminated by the UV light reaching the substrate 14′ through the openings 34 in the mask 32 are developed, cured or reacted (as appropriate for the type of photoresist employed) by interaction with the UV light falling thereon. Thus, the dimensions and locations of the apertures or holes 34 in the mask 32, as well as the position of the mask 32 relative to the substrate structure 14′, thereby define the localised areas or portions of the resist layer 24 which will be illuminated by the UV light and thus will be cured or reacted or developed so as to remain in place thereon-as localised non-conductive “islands” 40 as shown in FIG. 6-after removal of the remaining non-illuminated parts/portions/areas of the resist layer 24 upon the further processing of the arrangement.

Of course, as an alternative to a UV-based system, other wavelengths of light and other types of photocurable or photoreactable resist layer material 24 may be used instead, examples of which are well-known in the relevant art of photoresists.

After washing of the non-illuminated parts of the resist layer 24, there are obtained localised non-conductive islands 40 superimposed on the conductive layer 22 of the substrate structure 14′, as shown in FIG. 6.

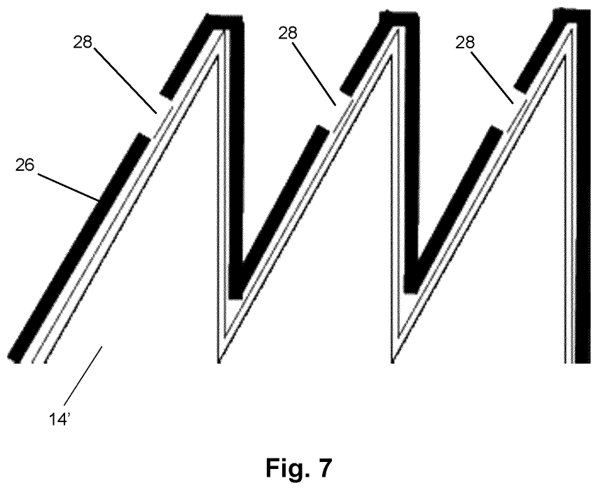

Next, the substrate structure 14′ of FIG. 6—which has thus been partially and selectively coated on the interiors of its concavities 12′ with the localised non-conductive island layers 40 on top of an otherwise substantially complete and continuous layer 22 (i.e. overlying substantially the whole of the upper surfaces of the concavities 12′ in the substrate 14′) of electrically conductive material (which again bridges the boundaries between adjacent concavities 12′, like the arrangement of the first embodiment of FIG. 2)—is then subjected to the same kind of galvanoplasty process as in the above “Production step 4” as used for the first embodiment of FIGS. 1 to 3, but in which modified embodiment as here in FIG. 6 the electrically conductive layer 22, but not the non-conductive islands 40, is galvanically plated with one or more layers of a metal which is to form and thus build up the actual walls 26 of each resulting microneedle—as shown in FIG. 7.

As shown in FIG. 7, the portions of the overall surface of the substrate 14′ where the metal microneedle-forming material is not so galvanically deposited correspond to the localised non-conductive islands 40, which therefore define and form the final holes or apertures 28 in the respective microneedle walls 26, which in this embodiment are located part-way up each microneedle's sidewall 26, and not at their tips. Of course by suitable design and positioning of the mask 32 used in the photoresist step as per FIG. 5, the finally produced holes 28 in the respective microneedles' sidewalls 26 may be designed to be located wherever (and to be shaped and configured however) they may be needed or desired, according to the particular design and end-use of the microneedles to be produced.

Once produced, the array, patch or chip of microneedles 26 of FIG. 7 may be subjected to any of the same post-production steps, such as the passivation step of “Production step 5” described above, or any further or alternative post-production treatment steps, as may be desired or appropriate.

Throughout the description and claims of this specification, the words “comprise” and “contain” and linguistic variations of those words, for example “comprising” and “comprises”, mean “including but not limited to”, and are not intended to (and do not) exclude other moieties, additives, components, elements, integers or steps.

Throughout the description and claims of this specification, the singular encompasses the plural unless expressly stated otherwise or the context otherwise requires. In particular, where the indefinite article is used, the specification is to be understood as contemplating plurality as well as singularity, unless expressly stated otherwise or the context requires otherwise.

Throughout the description and claims of this specification, features, components, elements, integers, characteristics, properties, compounds, chemical moieties or groups described in conjunction with a particular aspect, embodiment or example of the invention are to be understood to be applicable to any other aspect, embodiment or example described herein unless incompatible therewith or expressly stated otherwise.

Furthermore, it is expressly envisaged in this disclosure of the present invention that the various aspects, embodiments, examples, features and alternatives, and in particular the individual constructional, configurational or operational features thereof, set out in the preceding paragraphs, in the claims and/or in the following description and accompanying drawings, may be taken independently or in any combination of any number of same. For example, individual features described in connection with one particular embodiment, or described singly or in combination with another feature in any one or more embodiments, are applicable on their own or in combination with one or more other features to all embodiments and may be found and used in combination with any other feature in any given embodiment, unless expressly stated otherwise or such features are incompatible.

1. A method for the manufacture of one or more microneedles, the method comprising:
(i) providing a substrate structure of electrically insulating material including one or more concavities extending into the body of the substrate structure from a face thereof, the one or more concavities being internally shaped and configured so as to correspond to the external shape and configuration of the respective one or more microneedles to be formed;
(ii) applying selectively to at least one first surface or surface portion of each of the said one or more concavities a layer of an electrically conductive material; and
(iii) using a galvanoplastic technique, depositing on each layer of electrically conductive material in the said one or more concavities at least one layer or body of microneedle wall-forming material so as build up a wall of a respective microneedle in each respective said concavity.

2. A method according to claim 1, which is for the manufacture of one or more hollow microneedles, wherein the microneedle(s) each have a definable channel or bore extending internally therewithin from a base end thereof to an opposite tip portion thereof, for allowing passage therethrough of a liquid composition to be delivered by the or each respective microneedle upon use.

3. A method according to claim 1, which is for the manufacture of one or more substantially solid microneedles, wherein the microneedle(s) each have a substantially solid body structure, optionally with open pores or voids or a plurality of micro/nano-channels therewithin and/or an outer coating/carrier surface, for transporting a liquid composition to be delivered by the or each respective microneedle upon use from a base end thereof to an opposite tip portion thereof via said pores/voids/channels and/or by virtue of passage over the said outer coating/carrier surface.

4. A method according to claim 1, wherein the method includes a preliminary step, prior to step (i), of providing a master substrate including one or more convexities protruding from a face thereof, the one or more convexities being externally shaped and configured so as to correspond to the desired or intended internal shape and configuration of the respective one or more concavities, and transferring the shape and configuration of the said convexities on the master substrate to the body of electrically insulating material of the said substrate structure so as to form the substrate structure that is then used in step (i) of the method.

5. A method according to claim 1, wherein the application step (ii) comprises either:
(a) applying selectively to the said at least one first surface or surface portion of each of the said one or more concavities a composition comprising or containing the said electrically conductive material or a precursor of the said electrically conductive material, or
(b) applying selectively to the said at least one first surface or surface portion of each of the said one or more concavities the said electrically conductive material by a vacuum deposition technique;
optionally wherein the composition is aqueous or comprises a solvent or dispersant which is substantially aqueous or hydrophilic.

6. (canceled)

7. A method according to claim 1, wherein the step (ii) includes or inherently/implicitly comprises, in addition to the applying selectively to the at least one first surface or surface portion of each of the said one or more concavities the layer of the electrically conductive material, an action or step or implicit/inherent effecting of not applying the said electrically conductive material to at least one second surface or surface portion of each of the said one or more concavities, wherein the said second surface(s) or surface portion(s) is/are different from the said first surface(s) or surface portion(s) of each concavity;
optionally wherein, in order to achieve the said non-application of the electrically conductive material to the at least one second surface or surface portion of each concavity, the said at least one second surface or surface portion of each concavity has been formed or pre-treated so as to include or present a surface which substantially prevents adherence or bonding thereto of the electrically conductive material or a composition used to deliver or apply same;
and further optionally wherein the said formed or pre-treated at least one second surface or surface portion of each concavity comprises a textured surface or surface portion, optionally a surface/surface portion which comprises an array or series or arrangement of a plurality of ridges, protrusions, protuberances, grooves, channels, indentations or other surface formations which act to substantially prevent adherence or bonding thereto of the electrically conductive material (or a composition used to deliver or apply same) which is applied to the concavities' first surface(s)/surface portion(s), optionally wherein the textured surface or surface portion of the at least one second surface or surface portion of each concavity has or exhibits hydrophobic or superhydrophobic properties.

8. (canceled)

9. (canceled)

10. A method according to claim 7, wherein the method includes a preliminary step, prior to step (i), of providing a master substrate including one or more convexities protruding from a face thereof, the one or more convexities being externally shaped and configured so as to correspond to the desired or intended internal shape and configuration of the respective one or more concavities, and transferring the shape and configuration of the said convexities on the master substrate to the body of electrically insulating material of the said substrate structure so as to form the substrate structure that is then used in step (i) of the method,
and wherein the provision of the textured at least one second surface or surface portion of each concavity is effected by virtue of that/those second surface(s) or surface portion(s) having been formed by correspondingly externally shaped and configured portion(s) of the respective one or more convexities of the master substrate that has been used in the said preliminary step for forming the substrate structure used in step (i) of the method by transferring to the body of electrically insulating material thereof the shape and configuration of the said convexities on the master substrate.

11. A method according to claim 7, wherein, in order to achieve the said non-application of the electrically conductive material to the at least one second surface or surface portion of each concavity, the said at least one second surface or surface portion of each concavity has been formed or pre-treated so as to include or present a surface which substantially prevents adherence or bonding thereto of the electrically conductive material or a composition used to deliver or apply same,
and wherein the said formed or pre-treated at least one second surface or surface portion of each concavity comprises or has been provided or pre-treated with a layer of a material which has or exhibits hydrophobic or superhydrophobic properties.

12. A method according to claim 7, wherein, in order to achieve the said non-application of the electrically conductive material to the at least one second surface or surface portion of each concavity, the said at least one second surface or surface portion of each concavity has been formed or pre-treated so as to include or present a surface which substantially prevents adherence or bonding thereto of the electrically conductive material or a composition used to deliver or apply same,
and wherein the method includes, either immediately before or immediately after or simultaneously with the application step (ii), an auxiliary method step of applying selectively to the said at least one second surface or surface portion of each of the said one or more concavities a layer of an electrically non-conductive or electrically insulating material.

13. A method according to claim 7, wherein each said second surface or surface portion of each of the said one or more concavities corresponds to a respective tip portion of the respective microneedle to be formed in the said respective concavity, whereby each microneedle is formed with a tip portion including a hollow or open mouth or void or nozzle-like opening, i.e. where the microneedle walls have not been deposited by the galvanoplasty step (iii) selectively in that tip portion/region of the respective concavity of the substrate structure.

14. A method according to claim 7, wherein each said second surface or surface portion of each of the said one or more concavities corresponds to a respective sidewall portion of the respective microneedle to be formed in the said respective concavity, wherein the respective sidewall portion is a portion of a wall of the respective microneedle located between its tip portion and its opposite base end, whereby each microneedle is formed with a sidewall portion including a hollow or open mouth or void or opening, i.e. where the microneedle walls have not been deposited by the galvanoplasty step (iii) selectively in that sidewall portion/region of the respective concavity of the substrate structure.

15. A method according to claim 14, wherein the said application step (ii) is modified so as to comprise the following sequence of sub-steps:
(iia) applying to at least one first surface of each of the said one or more concavities a layer of an electrically conductive material, especially wherein the at least one said first surface comprises substantially the whole of or at least a majority of the internal surface area of each concavity;
(iib) applying an electrically non-conductive optical resist layer over the applied layer of electrically conductive material on the said at least one first surface;
(iic) using a prefabricated mask with one or more predefined openings therein which allow passage therethrough of light of a wavelength designed to develop or cure or photoreact with the resist layer to anchor or secure or bond or unite it to or with the electrically conductive layer therebeneath selectively only in those one or more regions or portions or areas thereof which have been illuminated by the light having passed through the respective opening(s) in the mask; and
(iid) removing the material of the resist layer only in those one or more regions or portions or areas thereof which have not been so developed or cured or photoreacted by the above step (iic), so as to leave in place, anchored or secured or bonded or united to or with the electrically conductive layer therebeneath, one or more selected isolated remnant regions or portions or areas of the electrically non-conductive optical resist layer, whereby said one or more selectively formed remnant regions or portions or areas of the electrically non-conductive optical resist layer constitute the above-defined at least one second surface or surface portion of each of the said one or more concavities which has or exhibits electrically non-conductive properties and so does not permit galvanic electrodeposition thereon of the microneedle wall material in the subsequent galvanoplasty step (iii) of the method.

16. A method according to claim 1, wherein the microneedle wall-forming material that is deposited in the galvanoplasty step (iii) comprises a metal, optionally nickel (Ni), further optionally one or more other electro-depositable metals selected from Cr, Cu and Ag.

17. A method according to claim 1, wherein the method is used to manufacture or form hollow microneedles, and wherein the microneedle wall-forming material that is deposited in the galvanoplasty step (iii) is so applied or deposited in one or more discrete layer-forming steps so as to build up the microneedle walls so as to have an average overall wall thickness in the range of from about 0.01 up to about 1 mm.

18. A method according to claim 1, which is for the manufacture of one or more substantially solid microneedles, wherein the microneedle(s) each have a substantially solid body structure, optionally with open pores or voids or a plurality of micro/nano-channels therewithin and/or an outer coating/carrier surface, for transporting a liquid composition to be delivered by the or each respective microneedle upon use from a base end thereof to an opposite tip portion thereof via said pores/voids/channels and/or by virtue of passage over the said outer coating/carrier surface,
and wherein the galvanoplasty step (iii) is designed so as to apply or deposit the microneedle wall-forming material (optionally with the said pores/voids/channels formed inherently within its structure) in a suitable number of layer-or body-forming steps so as to build up the or each respective substantially solid microneedle body structure from its outside towards its interior.

19. A method according to claim 1, wherein each said first surface or surface portion of each of the said one or more concavities comprises a major part of the respective concavity surface, which major part extends circumferentially or peripherally substantially completely around the respective concavity, whereby that major part of the respective concavity surface corresponds to a respective major sidewall portion of the respective microneedle to be formed in that respective concavity.

20. A method according to claim 1, wherein the method includes a post-galvanoplasty step, subsequent to the depositing of the walls of the respective microneedles in step (iii), of removing the thus formed one or more microneedles from the substrate structure.

21. A method according to claim 1, wherein the method includes a step, subsequent to the galvanoplasty step (iii) and removal of the thus formed one or more microneedles from the substrate structure, of subjecting the outer walls of the thus formed one or more microneedles to a passivation or other surface-treatment step, in order to render them compatible with or safe to use on a recipient's or subject's skin;
optionally wherein the said passivation step comprises subjecting the outer walls of the microneedles to an “atomic layer deposition” (ALD) process, or other vapour deposition process.

22. A method according to claim 1, wherein the method is used to manufacture or form a plurality of microneedles substantially simultaneously in the form of an array, patch or chip comprising said plurality of microneedles protruding from a major face thereof,
optionally wherein the longitudinal axes of the plurality of microneedles are all substantially parallel with one another, whereby all the microneedles point in substantially the same direction from the said major face of the array, patch or chip,
and wherein the substrate structure includes a plurality of the said concavities and the application step (ii) comprises applying to at least one first surface or surface portion of each respective one of the plurality of concavities a respective said layer of the electrically conductive material, so that in the galvanoplastic deposition step (iii) a respective layer or body of the microneedle wall-forming material is deposited on each one of the respective layers of electrically conductive material on the respective concavities' first surfaces or surface portions, so as to build up respective walls of the respective microneedles in the respective concavities.

23. A method according to claim 22, wherein the plurality of microneedles are joined together at their respective base ends by a unifying base or root or bridging portion extending commonly between all the said microneedles of the plurality,
and wherein in the application step (ii) the at least one first surface or surface of each of the said one or more concavities, on which is/are applied the said respective layers of electrically conductive material, extend across defining boundaries between adjacent concavities and/or extend from one concavity into an adjacent concavity;
optionally wherein the plurality of microneedles are joined together at their respective base ends by respective unifying bridging base portions extending between adjacent microneedles, and the method is such as to produce an array or chip with the bridging base portions having a greater thickness and thus strength as compared with the thickness of the walls of the microneedles themselves,
wherein the galvanoplasty step (iii) of the method comprises a plurality of galvanoplasty stages, in which at least one first galvanoplasty stage is carried out so as to deposit at least one first layer or body of microneedle wall-forming material in order to form at least the walls of the microneedles and the respective bridging base portions between them, and subsequent thereto at least one second, but selective, galvanoplasty stage is then carried out to selectively deposit on the said respective bridging base portions only at least one second layer or body of microneedle wall-forming material in order to increase the thickness of those respective bridging base portions as compared with the thickness of the microneedle walls,
optionally wherein the said selective deposition of the said at least one second layer or body of microneedle wall-forming material on the said respective bridging base portions only is achieved by application, after the first galvanoplasty stage, to those portions of the growing microneedle array or chip other than the bridging base portions—optionally onto at least the microneedles' wall portions themselves—a temporary coating or filler or masking layer of an electrically non-conductive material which substantially prevents any further build-up of the microneedles' wall thickness in those wall portions themselves, but only allows such build-up of the wall thickness in the bridging base portions only during the at least one second galvanoplasty stage.

24. (canceled)

25. A substrate structure for use in the method of claim 1, the substrate structure comprising a body of electrically insulating material including one or more concavities extending into the body thereof from a face thereof, and the one or more concavities being internally shaped and configured so as to correspond to the external shape and configuration of the respective one or more microneedles to be formed in the said method, wherein at least one first surface or surface portion of each of the said one or more concavities has applied thereto a layer of an electrically conductive material;
optionally wherein each of the said one or more concavities includes at least one second surface or surface portion thereof which is/are different from the said first surface(s) or surface portion(s) thereof, and each said second surface or surface portion of each concavity has been formed or pre-treated so as to include or present a surface which substantially prevents adherence or bonding thereto of the electrically conductive material (or a composition used to deliver or apply or form same) during the application step (ii) of the method.

26. (canceled)

27. One or more microneedles, or an array, patch or chip comprising one or more microneedles, the one or more said microneedles being formed by a method according to claim 1.
